(19) United States
(12) Patent Application Publication
(10) Pub. No.: US 2021/0271924 A1
(43) Pub. Date: Sep. 2, 2021

(54) ANALYZER, ANALYSIS METHOD, AND ANALYSIS PROGRAM

(71) Applicant: Hitachi, Ltd., Tokyo (JP)

(72) Inventors: Shinji TARUMI, Tokyo (JP); Wataru TAKEUCHI, Tokyo (JP); Georgios CHALKIDIS, Tokyo (JP); Shuntaro YUI, Tokyo (JP)

(21) Appl. No.: 17/140,455

(22) Filed: Jan. 4, 2021

(30) Foreign Application Priority Data
Feb. 28, 2020 (JP) 2020-033769

(51) Int. Cl. G06K 9/62; G06F 17/18; G06N 3/08; G16H 10/60

(52) U.S. Cl. CPC G06K 9/6218; G06K 9/6269; G06K 9/6232; G16H 10/60; G06F 17/18; G06N 3/08; G06K 9/6215

(57) ABSTRACT

An analyzer calculates a first feature amount data group from an intermediate layer by inputting each training data of a training data group into a learning model which includes an input layer, one or more intermediate layers, and an output layer, and is learned based on the training data group assigned to the input layer and a correct answer data group assigned to the output layer. A second feature amount data is calculated from the intermediate layer by inputting prediction target data of the learning model. A search processing of searching specific first feature amount data similar to the second feature amount data is calculated by the second calculation processing, from the first feature amount data group, and an extraction processing of extracting, from the training data group, specific training data, which is a calculation source of the specific first feature amount data searched by the search processing.

### CLAIM OF PRIORITY

The present application claims priority from Japanese patent application JP 2020-033769 filed on Feb. 28, 2020, the content of which is hereby incorporated by reference into this application.

### BACKGROUND OF THE INVENTION

### 1. Field of the Invention

The present invention relates to an analyzer, an analysis method, and an analysis program for analyzing data.

### 2. Description of the Related Art

There is a need to realize effective and efficient diagnosis support and healthcare related services that utilize real-world healthcare data. Healthcare in the related art is implemented in accordance with unified guidelines based on clinical knowledge, but it has been reported that this knowledge is based only on clinical studies of 10% or less of all cases, and it has not been possible to realize the optimal individual healthcare that should be.

Therefore, it is expected to realize a technique that supports service providers such as doctors and healthcare instructors based on real-world healthcare data analysis to provide optimal healthcare services. In particular, attention is focused on techniques for evaluating and predicting the effects and qualities of medical services (medication, lifestyle guidance, nursing care services, or the like) provided to individuals by using actual data. For example, the following techniques are disclosed.

JP-A-2014-71592 describes that “when a notice of medication completion indicating medication completion to a patient is transmitted from an electronic medical chart server 16, a medication effect information transmission device 17 collects medication effect information relating to medication effects represented by patients due to the medication from an image server 14 and the electronic medical chart server 16. The medication effect information collected by the medication effect information transmission device 17 is stored in a medication effect information database 54. When the medication effect information server 55 is searched by a client terminal 18 based on drug names and attribute information of a patient, the medication effect information server 55 transmits average medication effect information indicating an average medication effect of the searched drug to the client terminal 18. The client terminal 18 displays the average medication effect information in a time series on a monitor.”

WO 2012/080906 describes “a non-transitory computer-readable storage medium storing a set of instructions executable by a processor is provided. The set of instructions is operable to receive a current patient data set relating to a current patient, compare the current patient data set with a plurality of previous patient data sets (each corresponding to a previous patient), select one of the previous patient data sets based on a level of similarity between the selected previous patient data set and the current patient data set, and provide the selected previous patient data set to a user.”

Since a frequency of an implementation of intervention means such as medical services differs depending on the type, it is not easy to analyze the effect of the medical service using the intervention means as training data when there are few past examples. In particular, this problem is remarkable while analyzing the effect of combining a plurality of medical services, and there may be zero examples that completely match the combination. Such a problem can occur not only in the medical services but also in other services.

### SUMMARY OF THE INVENTION

An object of the invention is to improve analysis accuracy regardless of differences with past training data or the number of implementations.

An analyzer, which is an aspect of the invention disclosed in the present application, is an analyzer including a processor configured to execute a program and a storage device configured to store the program. The analyzer executes a first calculation processing of calculating a first feature amount data group from an intermediate layer by inputting each training data of a training data group into a learning model which includes an input layer, one or more intermediate layers, and an output layer, and is learned based on the training data group assigned to the input layer and a correct answer data group assigned to the output layer, a second calculation processing of calculating second feature amount data from the intermediate layer by inputting prediction target data of the learning model, a search processing of searching specific first feature amount data similar to the second feature amount data calculated by the second calculation processing, from the first feature amount data group calculated by the first calculation processing, and an extraction processing of extracting, from the training data group, specific training data, which is a calculation source of the specific first feature amount data searched by the search processing.

According to typical embodiments of the invention, the analysis accuracy can be improved regardless of differences with past training data or the number of implementations. Problems, configurations, and effects other than those described above will become apparent from the following description of embodiments.

### BRIEF DESCRIPTION OF THE DRAWINGS

FIG. 1 is an explanatory diagram showing an example of healthcare data analysis by an analyzer according to a first embodiment.

FIG. 2 is a block diagram showing an example of a hardware configuration of the analyzer.

FIG. 3 is an explanatory diagram showing an example of unshaped healthcare information.

FIG. 4 is a flowchart showing an example of a learning model generation processing procedure by the analyzer according to the first embodiment.

FIG. 5 is an explanatory diagram showing an example of shaped healthcare information.

FIG. 6 is an explanatory diagram showing an example of a neural network.

FIG. 7 is an explanatory diagram showing another example of the neural network.

FIG. 8 is a flowchart showing an example of a feature amount information generation processing procedure.

FIG. 9 is an explanatory diagram showing an example of prediction target unshaped healthcare information according to the first embodiment.

FIG. 10 is an explanatory diagram showing an example of prediction target shaped healthcare information according to the first embodiment.

FIG. 11 is a flowchart showing an example of an analysis processing procedure by the analyzer according to the first embodiment.

FIG. 12 is an explanatory diagram showing an example of results of a statistical processing (step S110).

FIG. 13 is an explanatory diagram showing an example of a cluster according to a second embodiment.

FIG. 14 is an explanatory diagram showing a generation example of a prediction model using the cluster shown in FIG. 13.

FIG. 15 is a flowchart showing an example of a prediction model generation processing procedure by an analyzer according to the second embodiment.

FIG. 16 is a flowchart showing an example of a prediction processing procedure by the analyzer according to the second embodiment.

FIG. 17 is an explanatory diagram showing an example of prediction target unshaped healthcare information according to the second embodiment.

FIG. 18 is an explanatory diagram showing an example of prediction target shaped healthcare information according to the second embodiment.

### DESCRIPTION OF THE PREFERRED EMBODIMENTS

### First Embodiment

### <Example of Healthcare Analysis>

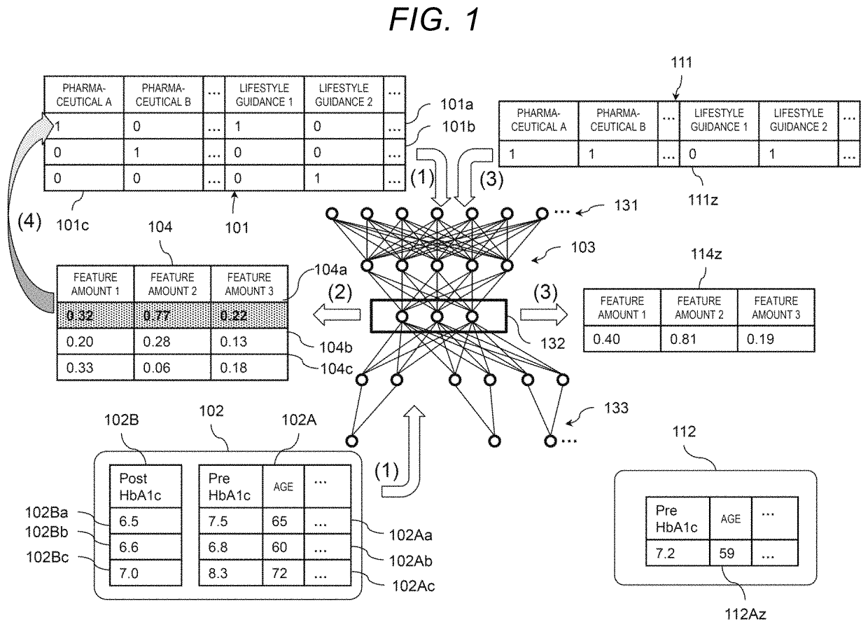

FIG. 1 is an explanatory diagram showing an example of healthcare data analysis by an analyzer according to a first embodiment. (1) The analyzer acquires a combination of intervention means information 101, which is training data, patient background information 102A and intervention effect information 102B, which is correct answer data 102. The intervention means information 101 is information indicating intervention means and is a set of intervention means data. Each record of the intervention means information 101 is intervention means data of each patient. In FIG. 1, the intervention means information 101 is configured by intervention means data 101a, 101b, and 101c of three patients a to c.

Intervention means is a medical service (medication, lifestyle guidance, nursing care service, or the like) intervened in an object person (for example, a patient or a test subject). “Intervention” means actions of implementing health guidance, assistance, independence support, medication, surgical treatment, or the like for the purpose of health promotion, disease prevention, illness treatment, or the like to the object person. The intervention means data includes, for example, the presence or absence of administered medicines and the presence or absence of medical services such as lifestyle guidance (presence is “1” and absence is “0”). That is, the intervention means data defines a combination of one or more medical services provided to a patient.

The patient background information 102A is information indicating a background of a patient and is a set of patient background data of each patient. Each record of the patient background information 102A is patient background data of each patient. The patient background information 102A is configured by patient background data 102Aa, 102Ab, and 102Ac of the three patients a to c. “Pre HbA1c” indicates a value of HbA1c before an intervention.

The intervention effect information 102B is information indicating an intervention effect and is a set of intervention effect data. The intervention effect is a result of the intervention, for example, a therapeutic outcome. Each record of the intervention effect information 102B is intervention effect data of each patient. In FIG. 1, the intervention effect information 102B is configured by intervention effect data 102Ba, 102Bb, and 102Bc of the three patients a to c. “Post HbA1c” indicates a value of HbA1c (Hemoglobin A1C) after the intervention.

The analyzer learns by giving the intervention means information 101 (the training data) and the correct answer data 102 to an input layer 131 and an output layer 133 of a neural network 103 as a training data set to generate a learning model. The learning model is the neural network 103 (hereinafter, referred to as the learning model 103) in which learning parameters (weight parameters and biases. Hyper parameters may be included (hereinafter the same)) are set.

(2) The analyzer generates feature amount information 104. The feature amount information 104 is internal representations of the learning model 103. The feature amount information 104 is a set of feature amount data of each patient. The feature amount data is calculation results of each neuron configuring a specific intermediate layer 132 in one or more intermediate layers of the learning model 103. The feature amount data is points in a feature amount space of a dimension of a number of neurons. If the patient background data and the intervention effect data between two patients are similar, the feature amount data between the two patients is also similar.

In addition, in FIG. 1, neurons of the intermediate layer 132 are three, and therefore feature amount data including feature amounts 1 to 3 are calculated for each patient. For this reason, the feature amount information 104 is configured by feature amount data 104a, 104b, and 104c of the three patients a to c.

(3) The analyzer inputs prediction target intervention means data 111z of a patient z in prediction target intervention means information 111 to the learning model 103 to calculate feature amount data 114z of the patient z from the specific intermediate layer 132 of the learning model 103.

(4) The analyzer searches feature amount data similar to feature amount data calculated in (3) form the feature amount information 104 calculated in (2). The feature amount data is one-dimensional vectors having as many elements as the number of neurons configuring the specific intermediate layer (three elements of feature amounts 1 to 3 in FIG. 1), and therefore feature amount data in the feature amount information 104, in which an inter-vector distance from the feature amount data 114z in the feature amount space is within a predetermined distance, is feature amount data similar to the feature amount data 114z. In FIG. 1, the feature amount data 104a corresponds to feature amount data similar to the feature amount data 114 (hereinafter, referred to as similar feature amount data 104a). The analyzer acquires the intervention means data 101a of the same patient a which is a calculation source of the similar feature amount data 104a.

In this way, the fact that the feature amount data 114z and 104a are similar means that the prediction target intervention means data 111z of the patient z and the intervention means data 101a of the patient a are similar in terms of both the patient background and the intervention effect. While analyzing the intervention effect, since it is preferable to analyze cases of similar interventions in the past, the analyzer acquires the intervention means data 101a, the patient background information 102Aa, and the intervention effect information 102Ba of the patient a in which an intervention similar to the prediction target intervention means for the patient z has been performed.

Cases of similar interventions extracted by the analyzer are analyzed to analyze the effect and the validity of the prediction target intervention means data 111z on the patient z. In this embodiment, statistical information and estimated values relating to the intervention effect are provided based on the intervention effect information 102Ba, and statistical information and estimated values relating to the patient background are provided based on the patient background information 102Aa.

Incidentally, in the above-mentioned embodiment, the intervention means data 101a to 101c of the intervention means information 101 and the prediction target intervention means data 111z of the prediction target intervention means information 111 are exemplified as data strings indicating the suitability of a plurality of different types of medical services (pharmaceutical prescriptions and lifestyle guidance). The plurality of different types of the medical services are not limited to the pharmaceutical prescriptions and the lifestyle guidance, but may include, for example, treatments and surgeries, and may therefore be a combination of two or more types of pharmaceutical prescriptions, lifestyle guidance, treatments, and surgeries. In addition, it may also be a combination of two or more types of medical services other than these.

In addition, the intervention means data 101a to 101c of the intervention means information 101 and the prediction target intervention means data 111z of the prediction target intervention means information 111 may be data strings indicating the suitability of a plurality of service attributes in one type of medical service.

In addition, in the intervention means information 101 and the prediction target intervention means information 111, the service attributes include, as the pharmaceutical prescriptions, the presence or absence of a pharmaceutical A and a pharmaceutical B, and may include a service attribute such as “pharmaceutical A→pharmaceutical B” in which the “pharmaceutical A” is prescribed in the past but changed to “pharmaceutical B”. Accordingly, it is possible to specify the intervention means information 101 and the prediction target intervention means information 111 in detail.

### <Example of Hardware Configuration of Analyzer>

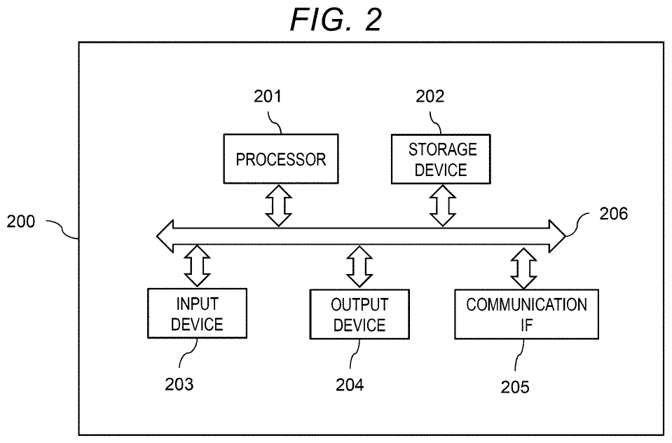

FIG. 2 is a block diagram showing an example of a hardware configuration of the analyzer. An analyzer 200 includes a processor 201, a storage device 202, an input device 203, an output device 204, and a communication interface (communication IF) 205. The processor 201, the storage device 202, the input device 203, the output device 204, and the communication IF 205 are connected via a bus 206. The processor 201 controls the analyzer 200. The storage device 202 is a work area of the processor 201. In addition, the storage device 202 is a non-temporary or temporary recording medium that stores various programs and data. The storage device 202 is, for example, a read only memory (ROM), a random access memory (RAM), a hard disk drive (HDD) or a flash memory. The input device 203 inputs data. The input device 203 is, for example, a keyboard, a mouse, a touch panel, a numeric keypad or a scanner. The output device 204 outputs data. The output device 204 is, for example, a display, a printer or a speaker. The communication IF 205 is connected to a network and transmits and receives data.

### <Unshaped Healthcare Information>

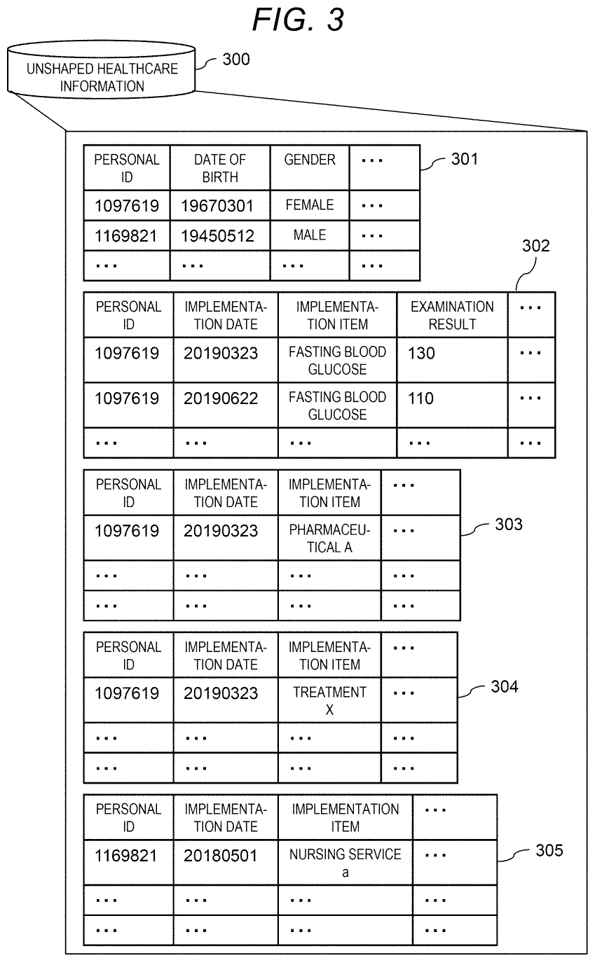

FIG. 3 is an explanatory diagram showing an example of unshaped healthcare information. Unshaped healthcare information 300 is stored in the storage device 202. In addition, the analyzer 200 may acquire the unshaped healthcare information 300 stored in another communicable computer via the communication IF 205.

The unshaped healthcare information 300 includes basic information 301, examination information 302, pharmaceutical information 303, treatment information 304, and related service information 305. The basic information 301 is basic information of a patient such as personal ID, date of birth, and gender. The personal ID is identification information that uniquely specifies a patient.

The examination information 302 is information relating to examinations such as a personal ID, implementation date of an examination for a patient specified by the personal ID, implementation items indicating contents of examinations, and examination results. The pharmaceutical information 303 is information relating to pharmaceuticals such as the personal ID, implementation date of using a pharmaceutical for a patient specified by the personal ID and implementation items indicating the used pharmaceutical. The treatment information 304 is information relating to treatments such as the personal ID, implementation date of a treatment for a patient specified by the personal ID and implementation items indicating contents of treatments. The related service information 305 is information relating to related services such as the personal ID, implementation date of related services for a patient specified by the personal ID and implementation items indicating the related services.

In the basic information 301, the examination information 302, the pharmaceutical information 303, the treatment information 304, and the related service information 305, a record with the same personal ID and the same implementation date is referred to as unshaped healthcare data. Since both the personal ID and the implementation date must be the same, if the implementation dates are different even for the same personal ID, they will be different unshaped healthcare data.

### <Learning Model Generation Processing>

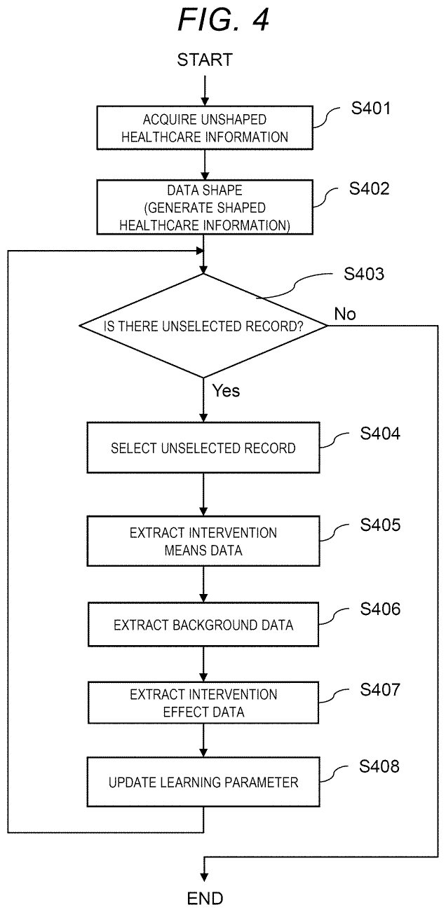

FIG. 4 is a flowchart showing an example of a learning model generation processing procedure by the analyzer 200 according to the first embodiment. The analyzer 200 acquires the unshaped healthcare information 300 from the storage device 202 or another communicable computer (step S401). Next, the analyzer 200 shapes the data of the unshaped healthcare information 300 to generate shaped healthcare information (step S402).

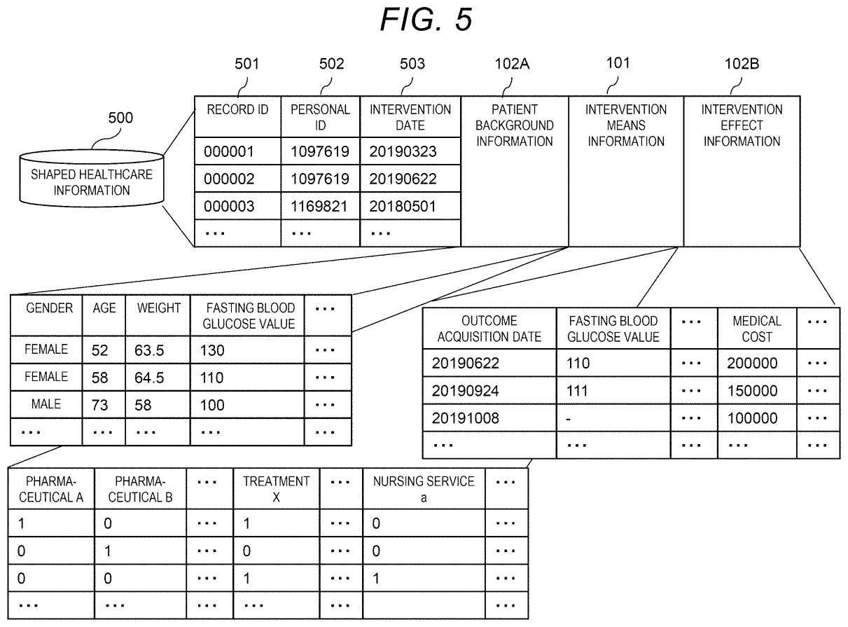

FIG. 5 is an explanatory diagram showing an example of shaped healthcare information. Shaped healthcare information 500 includes a record ID 501, a personal ID 502, an intervention date 503, the patient background information 102A, the intervention means information 101, and the intervention effect information 102B. Each record of the shaped healthcare information 500 is shaped healthcare data.

The record ID 501 is identification information that uniquely specifies shaped healthcare data. The personal ID 502 is a personal ID specified by the unshaped healthcare information 300. The intervention date 503 is a date of the intervention to a patient specified by the personal ID 502. The intervention date 503 is an implementation date of any one of the basic information 301, the examination information 302, the pharmaceutical information 303, the treatment information 304, and the related service information 305, which configure the unshaped healthcare information 300.

The patient background information 102A includes personal information such as, for example, gender, age, weight, a fasting blood glucose level. The patient background information 102A is shaped from, for example, the basic information 301. Each record of the patient background information 102A is referred to as patient background data.

The intervention means information 101 includes various intervention means such as, for example, the pharmaceutical A, the pharmaceutical B, treatment X, and nursing care service a. The intervention means information 101 is shaped from, for example, the pharmaceutical information 303, the treatment information 304, and the related service information 305. Each record of the intervention means information 101 is referred to as intervention means data.

The intervention effect information 102B includes various intervention effects such as, for example, outcome acquisition date, a fasting blood glucose level, and medical costs. The intervention effect information 102B is shaped from, for example, the examination information 302. Each record of the intervention effect information 102B is referred to as intervention effect data.

Return to FIG. 4, the analyzer 200 determines if there is unselected shaped healthcare data (step S403). If there is an unselected record (shaped healthcare data) (step S403: Yes), the analyzer 200 selects the unselected record (step S404) and extracts intervention means, background data, and intervention effect data from the selected record (steps S405 to S406).

The analyzer 200 gives the extracted intervention means data as training data and the extracted background data and intervention effect data as correct answer data to the neural network 103, updates learning parameters of the neural network 103 (step S408), and returns to step S403. If there is no unselected record (step S403: No), in the analyzer 200, the neural network 103 becomes the learning model 103 in which the latest learning parameters are set. Accordingly, the learning model generation processing ends.

### <Neural Network>

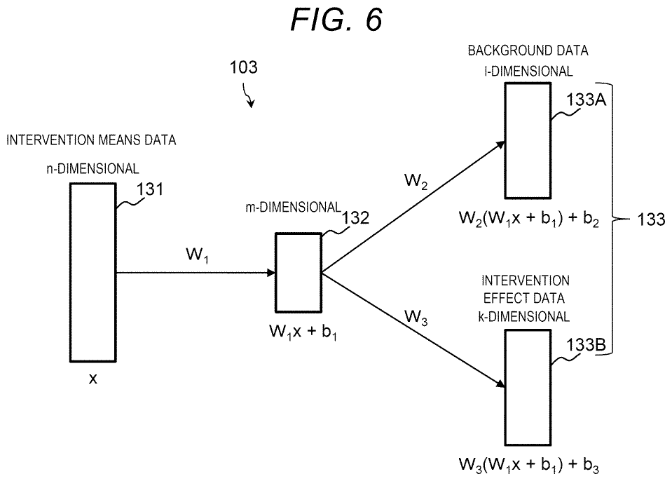

FIG. 6 is an explanatory diagram showing an example of the neural network 103. The neural network 103 is configured by the input layer 131, one intermediate layer 132, and the output layer 133 (133A and 133B). Intervention means data is input to the input layer 131. The intervention means data is an n-dimensional vector x.

The intermediate layer 132 has a weight parameter W1 and a bias b1 as learning parameters, and executes the operation of the following Formulation (1). Operation results of Formulation (1) become feature amount data shown in FIG. 1.

x2A=W1×x+b1 (1)

W1 is represented by the m×n matrix of the following Formulation (2). Of these, v1i(1≤i≤n) in Formulation (2) is an m-dimensional column vector. In addition, the bias b1 is also an m-dimensional column vector.

W1=(v11,v12,v13, . . . ,v1n) (2)

An execution result (x2A=W1×x+b1) of Formulation (1) is input to the first output layer 133A. The first output layer 133A has a weight parameter W2 and a bias b2 as learning parameters, and executes the operation of the following Formulation (3).

y1=W2×x2A+b2 (3)

W2 is represented by the l×n matrix of the following Formulation (4). Of these, v2i(1≤i≤1) in Formulation (4) is an 1-dimensional column vector. In addition, the bias b2 is also an 1-dimensional column vector.

W2=(v21,v22,v23, . . . ,v21) (4)

An execution result (x2A=W1×x+b1) of Formulation (1) is input to a second output layer 133B. The second output layer 133B has a weight parameter W3 and a bias b3 as learning parameters, and executes the operation of the following Formulation (5).

y2=W3×x2A+b3 (5)

W3 is represented by the k×n matrix of the following Formulation (6). Of these, v3i(1≤i≤k) in Formulation (6) is a k-dimensional column vector. In addition, the bias b3 is also a k-dimensional column vector.

W3=(v31,v32,v33, . . . ,v3k) (6)

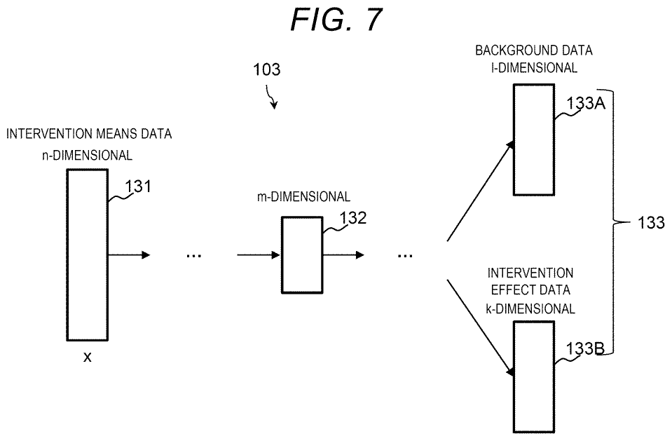

FIG. 7 is an explanatory diagram showing another example of the neural network 103. The intermediate layer of the neural network 103 in FIG. 6 is one layer, whereas the intermediate layer of the neural network in FIG. 7 has m layers. Similar to the specific intermediate layer 132 in FIG. 1, the specific intermediate layer 132 among the intermediate layers of the m layer generates the feature amount information 104.

### <Feature Amount Information Generation Processing>

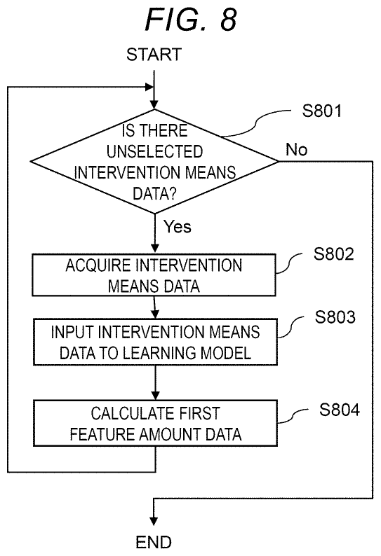

FIG. 8 is a flowchart showing an example of a feature amount information generation processing procedure. The analyzer 200 determines whether or not the shaped healthcare information 500 includes unselected intervention means data (step S801). If there is unselected intervention means data (step S801: Yes), the analyzer 200 acquires the unselected intervention means data from the shaped healthcare information 500 (step S802).

The analyzer 200 inputs acquired intervention means data to the learning model 103 (step S803). The analyzer 200 calculates feature amount data by the specific intermediate layer 132 of the learning model 103 and stores it in the storage device 202, and returns to step S801 (step S804). This calculated feature amount data is referred as first feature amount data for convenience.

If the intervention means data acquired in step S802 is the intervention means data 101a shown in FIG. 1, the feature amount data 104a is calculated as the first feature amount data. If there is no unselected intervention means data in step S801 (step S801: No), the feature amount information generation processing ends. In this way, the feature amount information 104 as shown in (2) of FIG. 1 is generated.

### <Prediction Target Unshaped Healthcare Information>

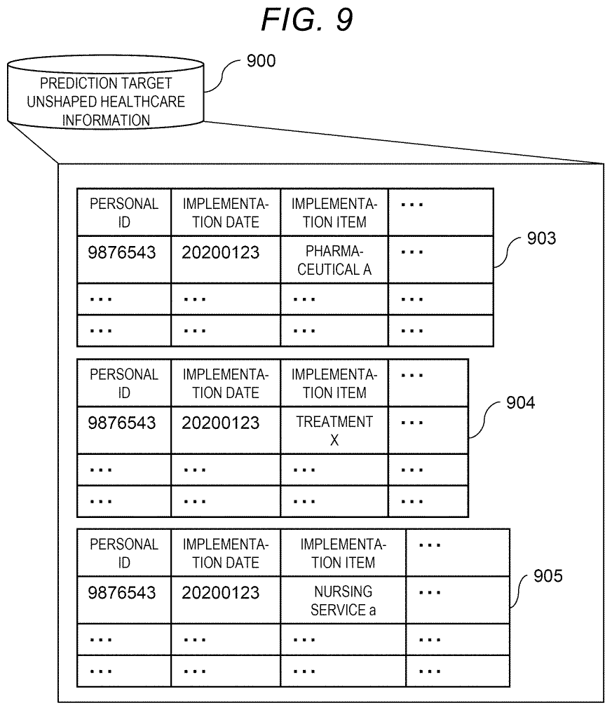

FIG. 9 is an explanatory diagram showing an example of prediction target unshaped healthcare information according to the first embodiment. Prediction target unshaped healthcare information 900 is stored in the storage device 202. In addition, the analyzer 200 may acquire the prediction target unshaped healthcare information 900 stored in another communicable computer via the communication IF 205.

The prediction target unshaped healthcare information 900 includes pharmaceutical information 903, treatment information 904, and related service information 905. The pharmaceutical information 903, the treatment information 904, and the related service information 905 include the same items as the pharmaceutical information 303, the treatment information 304, and the related service information 305 shown in FIG. 3. In addition, since examinations have not been implemented, examination information is not included in the prediction target unshaped healthcare information 900. When at least a part of the unshaped healthcare information 300 is used as the prediction target unshaped healthcare information 900, the basic information 301 and the examination information 302 may be excluded.

In the pharmaceutical information 903, the treatment information 904, and the related service information 905, a record with the same personal ID and the same implementation date is referred to as prediction target unshaped healthcare data. Since both the personal ID and the implementation date must be the same, if the implementation dates are different even for the same personal ID, they will be different prediction target unshaped healthcare data.

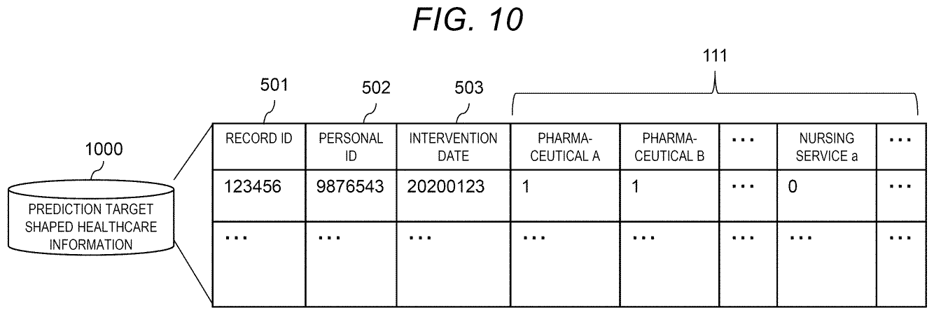

FIG. 10 is an explanatory diagram showing an example of the prediction target shaped healthcare information according to the first embodiment. Prediction target shaped healthcare information 1000 includes the record ID 501, the personal ID 502, the intervention date 503, and the prediction target intervention means information 111. Each record of the prediction target shaped healthcare information 1000 is prediction target shaped healthcare data.

### <Analysis Processing>

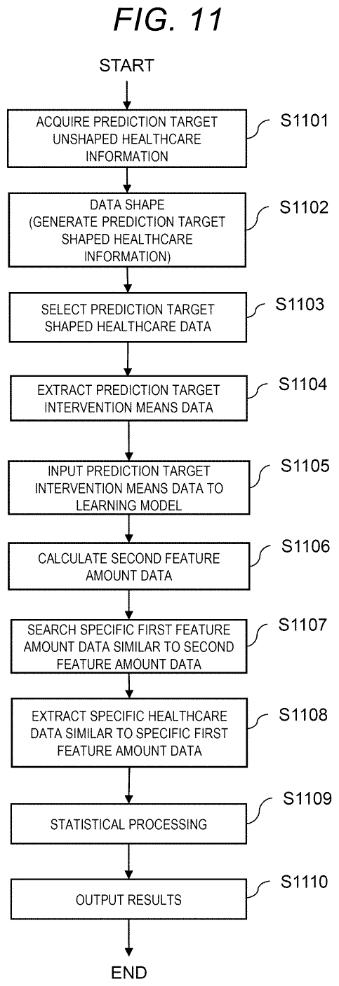

FIG. 11 is a flowchart showing an example of an analysis processing procedure by the analyzer 200 according to the first embodiment. The analyzer 200 acquires the prediction target unshaped healthcare information 900 from the storage device 202 or another communicable computer (step S1101). Next, the analyzer 200 shapes the data of the prediction target unshaped healthcare information 900 to generate the prediction target shaped healthcare information 1000 (step S1102).

The analyzer 200 selects the prediction target shaped healthcare data from the prediction target shaped healthcare information 1000 (step S1103). The analyzer 200 extracts the prediction target intervention means data from selected prediction target shaped healthcare information (step S1104).

In addition, the analyzer 200 may, by user operations, receive input of intervention means data such as “pharmaceutical A and pharmaceutical X” from the input device 203 or another communicable computer via the communication IF 205, instead of acquiring intervention means data as steps S1101 to S1104.

The analyzer 200 inputs extracted prediction target intervention means data to the learning model 103 (step S1105). The analyzer 200 calculates the feature amount data by the specific intermediate layer 132 of the learning model 103 (step S1106). This feature amount data is referred as second feature amount data for convenience to be distinguished from the first feature amount data in step S804. If the prediction target intervention means data acquired in step S1104 is the prediction target intervention means data 111z shown in FIG. 1, the feature amount data 114z is calculated as the second feature amount data.

The analyzer 200 searches specific first feature amount data similar to the second feature amount data (step S1107). Specifically, for example, the analyzer 200 calculates a similarity between each first feature amount data and the second feature amount data. If the similarity is equal to or greater than a similarity threshold value, the first feature amount data becomes the specific first feature amount data similar to the second feature amount data.

The similarity is, for example, a distance between the first feature amount data and the second feature amount data in the feature amount space. If the reciprocal of the calculated distance is equal to or greater than the similarity threshold value, the first feature amount data becomes the specific first feature amount data similar to the second feature amount data.

In addition, the similarity threshold value may be a value set in advance in the analyzer 200, or a value received from the input device 203 or another communicable computer via the communication IF 205 by user operations.

The analyzer 200 extracts specific healthcare data corresponding to the specific first feature amount data (step S1108). Specifically, for example, the analyzer 200 extracts the shaped healthcare data including the intervention means data, which is a calculation source of the specific first feature amount data, from the shaped healthcare information 500. In the example in (5) of FIG. 1, if the specific first feature amount data is assumed as the feature amount data 104a, the intervention means data 101a is extracted from the intervention means information 101 as the specific healthcare data.

After this, the analyzer 200 executes a statistical processing (step S1110) and outputs results of the statistical processing (step S1110) (step S1111). Details of the statistical processing (step S1110) will be described below.

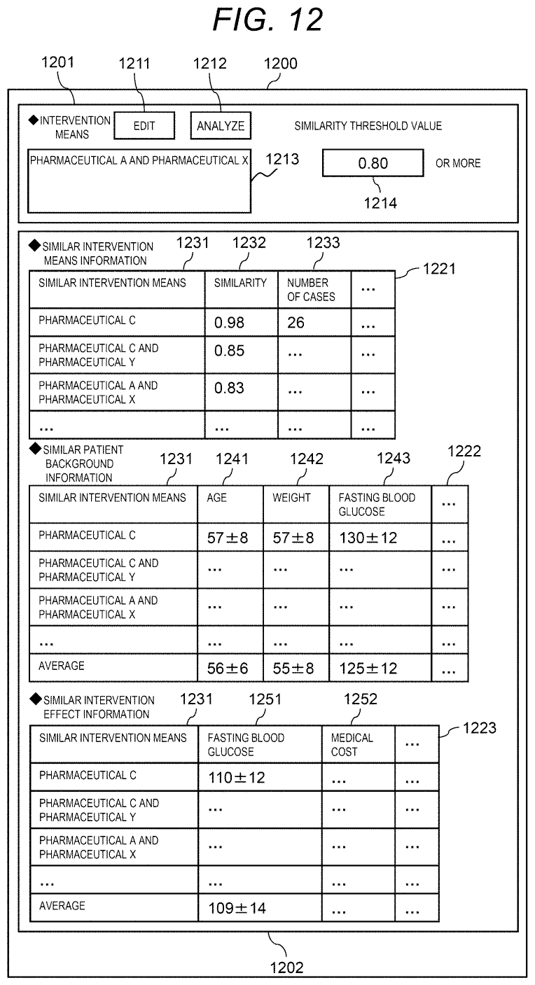

FIG. 12 is an explanatory diagram showing an example of the results of the statistical processing (step S1110). A result screen 1200 is a screen for displaying the results of the statistical processing (step S1110). The result screen includes an input region 1201 and an output region 1202.

The input region 1201 includes an edit button 1211, an analysis button 1212, an intervention means input column 1213, and a threshold value input column 1214. The edit button 1211 is a button that enables input of character strings in the intervention means input column 1213 and the threshold value input column 1214 by pressing. The analysis button 1212 is a button for executing the analysis processing shown in FIG. 11 by pressing.

The intervention means input column 1213 is an input column which receives input of the prediction target intervention means data 111z, such as “pharmaceutical A and pharmaceutical X” from the input device 203 or another communicable computer via the communication IF 205 by user operations. The threshold value input column 1214 is an input column which receives input of a numerical value showing the similarity, such as “0.80” from the input device 203 or another communicable computer via the communication IF 205 by user operations.

The output region 1202 includes similar intervention means information 1221, similar patient background information 1222, and similar intervention effect information 1223. The similar intervention means information 1221 includes information such as similarity 1232 and the number of cases 1233 for each similar intervention means 1231. The similar intervention means 1231 is intervention means data which is a calculation source of the specific first feature amount data (step S1107) similar to the second feature amount data calculated from the specific intermediate layer 132 in step S1106 as a result of the intervention means data input in the intervention means input column 1213 being input to the learning model 103, and is extracted in step S1108.

The similarity 1232 is a similarity between the second feature amount data calculated from the specific intermediate layer 132 in step S1106 and the similar intervention means 1231 (for example, the reciprocal of the calculated distance) as a result of the prediction target intervention means data input in the intervention means input column 1213 being input to the learning model 103, and is calculated in step S1107. Similar intervention means displayed as the similar intervention means information 1221 is, for example, intervention means data whose similarity 1232 is equal to or greater than the similarity threshold value.

The number of cases 1233 is a count value of the shaped healthcare data having the similar intervention means 1231 as the intervention means data, and is calculated by the statistical processing (step S1109).

The similar patient background information 1222 includes information such as age 1241, weight 1242, and fasting blood glucose 1243 for each similar intervention means 1231.

The age 1241 is a statistical value (for example, average value ±standard deviation) of one or more ages specified from the similar intervention means. Specifically, for example, the analyzer 200 specifies the shaped healthcare data (hereinafter, referred to as similar shaped healthcare data) including the intervention means data from the shaped healthcare information 500 for each intervention means data which becomes the similar intervention means 1231. The analyzer 200 extracts ages included in the patient background data in the similar shaped healthcare data, for each similar shaped healthcare data. The analyzer 200 calculates an average value and a standard deviation of ages extracted for each similar shaped healthcare data, and displays as the age 1241 corresponding to the similar intervention means 1231 of the similar patient background information 1222. The age 1241 is calculated by the statistical processing (step S1109).

The weight 1242, similar to the age 1241, is a statistical value (for example, average value ±standard deviation) of one or more weights specified from the similar intervention means 1231. Specifically, for example, the analyzer 200 extracts weights included in the patient background data in the similar shaped healthcare data, for each similar shaped healthcare data. The analyzer 200 calculates an average value and a standard deviation of extracted weights for each similar shaped healthcare data, and displays as the weight 1242 corresponding to the similar intervention means 1231 in the similar patient background information 1222. The weight 1242 is calculated by the statistical processing (step S1109).

The fasting blood glucose 1243, similar to the age 1241, is a statistical value (for example, average value ±standard deviation) of one or more fasting blood glucose values before the interventions specified by the similar intervention means 1231. Specifically, for example, the analyzer 200 extracts fasting blood glucose included in the patient background data in the similar shaped healthcare data, for each similar shaped healthcare data. The analyzer 200 calculates an average value and a standard deviation of the extracted fasting blood glucose for each similar shaped healthcare data, and displays as the fasting blood glucose 1243 corresponding to the similar intervention means 1231 in the similar patient background information 1222. The fasting blood glucose 1243 is calculated by the statistical processing (step S1109).

The similar intervention effect information 1223 includes information such as fasting blood glucose 1251 and medical cost 1252, for each similar intervention means 1231.

The fasting blood glucose 1251 is a statistical value (for example, average value ±standard deviation) of one or more fasting blood glucose values after the interventions specified from the similar intervention means 1231. Specifically, for example, the analyzer 200 extracts the fasting blood glucose included in the intervention effect data in the similar shaped healthcare data, for each similar shaped healthcare data. The analyzer 200 calculates an average value and a standard deviation of the extracted fasting blood glucose for each similar shaped healthcare data, and displays as the fasting blood glucose 1251 corresponding to the similar intervention means 1231 in the similar intervention effect information 1223. The fasting blood glucose 1251 is calculated by the statistical processing (step S1109).

The medical cost 1252, similar to the fasting blood glucose 1251, is a statistical value (for example, average value ±standard deviation) of one or more medical costs specified by the similar intervention means. Specifically, for example, the analyzer 200 extracts the medical costs included in the intervention effect data in the similar shaped healthcare data, for each similar shaped healthcare data. The analyzer 200 calculates an average value and a standard deviation of the extracted medical costs for each similar shaped healthcare data, and displays as the medical cost 1252 corresponding to the similar intervention means in the similar intervention effect information 1223. The medical cost 1252 is calculated by the statistical processing (step S1109).

As described above, according to a first embodiment, it is possible to provide a case of performing a similar intervention such as the similar intervention means information 1221, statistical information relating to the intervention effect of this case such as the similar intervention effect information 1223, and statistical information (for example, average value and standard deviation) relating to the patient background such as the similar background information 1222, from an intervention means data group that does not match the prediction target intervention means data 111z, which is a combination of one or more medical services provided to the patient z.

### Second Embodiment

Next, a second embodiment will be described. In the first embodiment, information of cases of similar interventions is provided, and thus the analyzer 200 provides a case of performing a similar intervention, statistical information relating to the intervention effect of this case, and statistical information (for example, average value and standard deviation) relating to the patient background. In the second embodiment, since similar intervention means data have similar intervention effects, the analyzer 200 generates a prediction model capable of making such predictions.

Then, the analyzer 200 can predict intervention effect data of the patient z by inputting the prediction target intervention means data 111z of the patient z and the prediction target patient background data 111Az of the patient z to this prediction model. Unless otherwise specified, the contents of FIGS. 1 to 10 described in the first embodiment are applied to the second embodiment. In addition, the same configurations as those in FIG. 1 are denoted by the same reference numerals, and the description thereof is omitted.

### <Cluster>

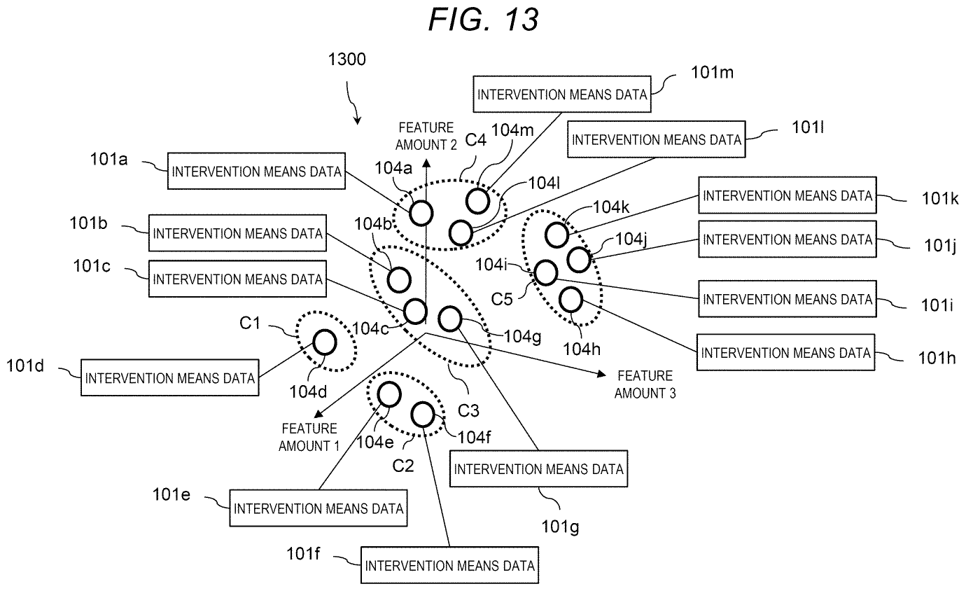

FIG. 13 is an explanatory diagram showing an example of a cluster according to the second embodiment. The feature amount data 104a to 104m of the feature amount space 1300 is calculated from the specific intermediate layer 132 as a result of inputting the intervention means data 101a to 101m into the learning model 103. Clusters C1 to C5 include similar feature amount data.

### <Prediction Model>

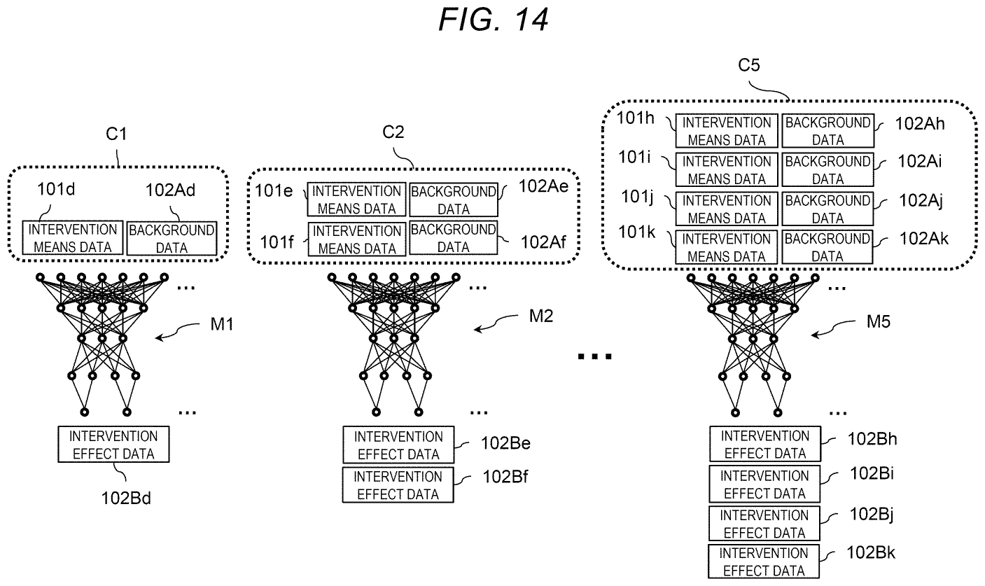

FIG. 14 is an explanatory diagram showing a generation example of a prediction model using the cluster shown in FIG. 13. The generation of prediction models M1 to M5 is executed in the same manner as the generation of the learning model 103. For example, in a case of a cluster C5, the analyzer 200 gives intervention means data 101h to 101k, which is a calculation source of feature amount data 104h to 104k belonging to the cluster C5, and patient background data 102Ah to 102Ak to an input layer of a neural network, gives corresponding intervention effect data 102Bh to 102Bk to an output layer to learn, and acquires learning parameters (weight parameters and biases).

When these learning parameters are set in the neural network, it becomes the prediction model M5. The prediction models M1 to M4 are also generated in the same way using clusters C1 to C4. Incidentally, the prediction models M1 to M5 may be other prediction analysis models, such as a linear regression model.

### <Generation of Prediction Model>

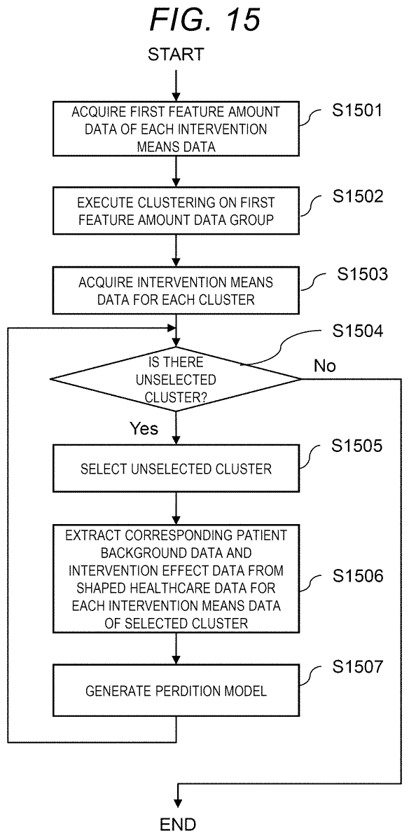

FIG. 15 is a flowchart showing an example of a prediction model generation processing procedure by the analyzer 200 according to the second embodiment. The analyzer 200 extracts each intervention means data from the shaped healthcare information 500 and inputs it into the learning model 103 to acquire feature amount data calculated from the specific intermediate layer 132 (step S1501). Accordingly, feature amount data 104a to 104m as shown in FIG. 13 is obtained.

The analyzer 200 executes clustering on a feature amount data group acquired in step S1501 (step S1502). For example, when a hierarchical clustering is performed, the analyzer 200 (1) sets individual feature amount data as one cluster, (2) calculates similarities between clusters and merges the most similar clusters, and (3) executes (2) until the number of clusters converges to a predetermined number. The similarity between the clusters in (2) is the reciprocal of a distance between clusters calculated by, for example, the nearest neighbor method, the farthest neighbor method, or the centroid method. Accordingly, the clusters C1 to C5 as shown in FIG. 13 are generated. In addition, the analyzer 200 may execute non-hierarchical clustering such as the k-means method to generate the clusters C1 to C5.

The analyzer 200 acquires the intervention means data which is the calculation source of the feature amount data belonging to the clusters, for each cluster (step S1503). For example, in the case of the cluster C5, the analyzer 200 acquires the intervention means data 101h to 101k, which is a calculation source of the feature amount data 104h to 104k belonging to the cluster C5.

Further, the analyzer 200 determines whether or not there is an unselected cluster in a cluster group (step S1504). When there is an unselected cluster (step S1504: Yes), the analyzer 200 selects the unselected cluster (step S1505).

The analyzer 200 extracts corresponding patient background data and intervention effect data from the shaped healthcare data, for each intervention means data of the selected cluster (step S1506). For example, in the case of the cluster C5, the analyzer 200 extracts the patient background data 102Ah to 102Ak corresponding to the intervention means data 101h to 101k and the intervention effect data 102Bh to 102Bk form healthcare data of patients h to k.

The analyzer 200 generates a prediction model for the selected cluster (step S1507) and returns to step S1504. For example, in the case of the cluster C5, the analyzer 200 gives the intervention means data 101h to 101k, which is a calculation source of the feature amount data 104h to 104k belonging to the cluster C5, to the input layer of the neural network, gives the patient background data 102Ah to 102Ak corresponding to the intervention means data 101h to 101k and the intervention effect data 102Bh to 102Bk to an output layer of the neural network, and generates the prediction model M5.

In step S1504, if there is no unselected cluster (step S1504: No), the prediction model generation processing ends. The generated prediction model group (for example, the prediction models M1 to M5) is stored in the storage device 202 or another communicable computer via the communication IF 205.

### <Prediction Processing>

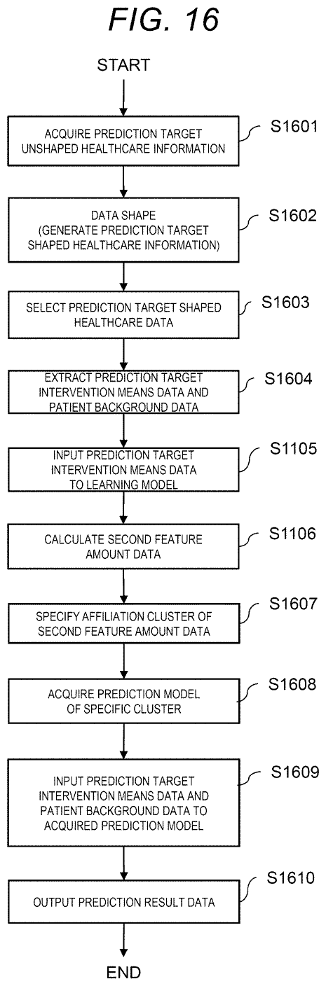

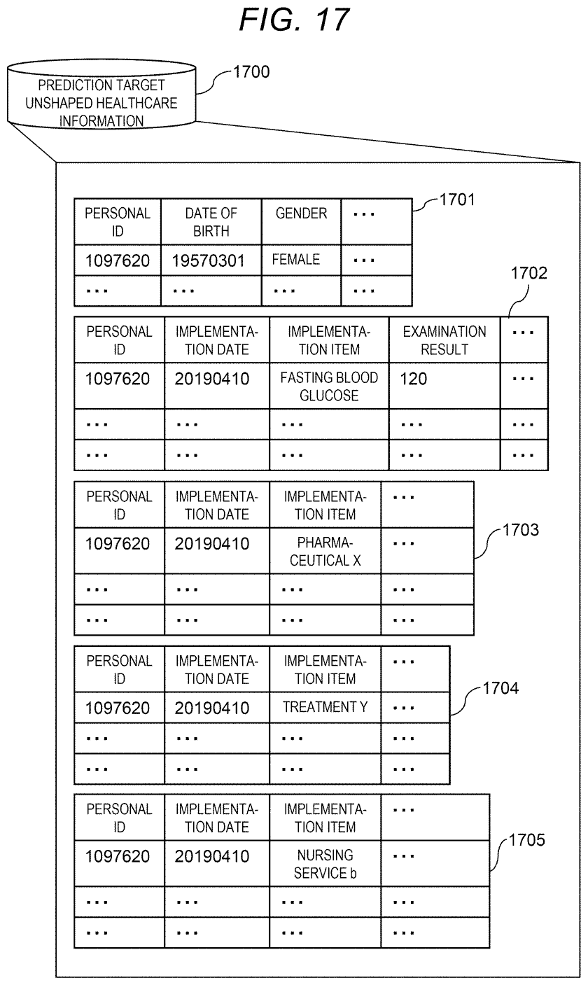

FIG. 16 is a flowchart showing an example of a prediction processing procedure by the analyzer 200 according to the second embodiment. FIG. 17 is an explanatory diagram showing an example of prediction target unshaped healthcare information according to the second embodiment. Prediction target unshaped healthcare information 1700 is stored in the storage device 202. In addition, the analyzer 200 may acquire the prediction target unshaped healthcare information 1700 stored in another communicable computer via the communication IF 205.

The prediction target unshaped healthcare information 1700 includes basic information 1701, examination information 1702, pharmaceutical information 1703, treatment information 1704, and related service information 1705. The basic information 1701 to the related service information 1705 is information similar to the basic information 301 to the related service information 305 as shown in FIG. 3.

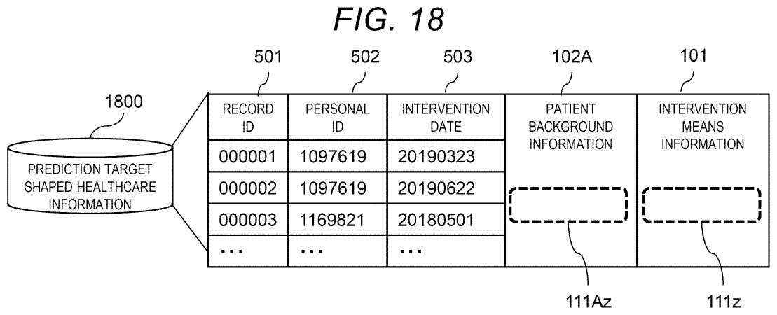

FIG. 18 is an explanatory diagram showing an example of prediction target shaped healthcare information according to the second embodiment. Prediction target shaped healthcare information 1800 includes the record ID 501, the personal ID 502, the intervention date 503, the patient background information 102A, and the intervention means information 101, similar to the shaped healthcare information 500 as shown in FIG. 5. However, since a prediction target is mainly described, the intervention effect information 102B is not included. The prediction target patient background data 111Az of the patient z is included in the patient background information 102A. The prediction target intervention means data 111z of the patient z is included in the intervention means information 101.

Returns to FIG. 16, the analyzer 200 acquires the prediction target unshaped healthcare information 1700 from the storage device 202 or another communicable computer (step S1601). Next, the analyzer 200 shapes data of the prediction target unshaped healthcare information 1700 to generate the prediction target shaped healthcare information 1800 (step S1602). The analyzer 200 selects the prediction target shaped healthcare data from the prediction target shaped healthcare information 1800 (step S1603). The analyzer 200 extracts the prediction target intervention means data and the prediction target patient background data from the selected prediction target shaped healthcare information (step S1604). Since steps S1105 and S1106 are processing similar to steps S1105 and S1106 on FIG. 11, descriptions thereof are omitted.

In addition, the analyzer 200 may, by user operations, receive input of the intervention means data and the patient background data, such as “pharmaceutical A and pharmaceutical X” and “SET age=79” from the input device 203 or another communicable computer via the communication IF 205, input the prediction target intervention means data and prediction target patient background data thereof to the learning model 103, and calculate a second feature amount data from the specific intermediate layer 132, instead of acquiring the intervention means data and patient background data as steps S1601 to S1604.

The analyzer 200 specifies a belonging cluster of the second feature amount data after step S1106 (step S1607). The belonging cluster of the second feature amount data is a cluster including the second feature amount data in the feature amount space. If there is no belonging cluster, the analyzer 200 may output the fact to prompt the reselection of the prediction target shaped healthcare data (or the re-input of the prediction target intervention means data), and may re-execute clustering so that the total number of clusters decreases until the second feature amount data is included.

The analyzer 200 acquires a prediction model of the cluster specified in step S1607 from a storage destination of the prediction model group (step S1608). The analyzer 200 outputs prediction result data from the prediction model by inputting the prediction target intervention means data and the prediction target patient background data extracted in step S1604 in the acquired prediction model (step S1610). The prediction result data includes prediction values of the intervention effect data. Accordingly, the prediction processing ends.

As described above, according to the second embodiment, the prediction model of the cluster to which the prediction target intervention means data corresponds is specified from the prediction models M1 to M5 generated for each of the clusters C1 to C5. Therefore, by using the specified prediction model, even if there is no intervention means data that matches the prediction target intervention means data, it is possible to obtain the prediction results of the intervention effect data based on the prediction model constructed from the prediction target intervention means data and the patient background data.

### Third Embodiment

A third embodiment will be described. Unless otherwise specified, the contents of FIGS. 1 to 12 described in the first embodiment and the contents of FIGS. 13 and 15 described in the second embodiment are applied to the third embodiment. In addition, the same configurations as those in the first embodiment and the second embodiment are denoted by the same reference numerals, and the description thereof is omitted.

In the second embodiment, the analyzer 200 generates the prediction models M1 to M5 for the respective clusters C1 to C5. In contrast, in the third embodiment, the analyzer 200 generates the clusters C1 to C5 as shown in FIG. 13 (step S1502), but extracts specific healthcare data (step S1108) to execute the statistical processing (step S1109) as in the first embodiment without generating the prediction models M1 to M5 (step S1507).

Specifically, for example, the analyzer 200 specifies an affiliation cluster of the second feature amount data from the clusters C1 to C5. For example, the analyzer 200 specifies a cluster most similar (the distance is close) to the second feature amount data by, for example, the above-mentioned nearest neighbor method, the farthest neighbor method, or the centroid method. Further, the analyzer 200 searches specific first feature amount data similar to the second feature amount data from the first feature amount data group in a specified affiliation cluster, as step S1107. Thereafter, the analyzer 200 executes steps S1108 to S1110 similar to the first embodiment.

In this way, by the third embodiment, since the affiliation cluster of the second feature amount data is specified by the similarity between the second feature amount data and the cluster, there is no need to calculate the similarity with each first feature amount data. Therefore, it is possible to improve the calculation processing accuracy.

Although the analysis using the medical services has been described in the first to third embodiments, the training data set used for the analysis is not limited to the medical institution origin, and may be widely related to health. For example, it may be data related to medical cost payment such as a medical fee detailed statement. Further, the invention is used for not only the medical services but also other services. For example, the invention may be applied to support services for sports competitions. In this case, the intervention means information 101 becomes an athlete's practice method or a nutritional supplement prescription, the patient background information 102A becomes athlete's background information, and the intervention effect information 102B becomes measurement results such as muscle strength and running ability.

In addition, the invention may be applied to a repair service of a machine tool. In this case, the intervention means information 101 becomes repair items, the patient background information 102A becomes background information of the machine tool (such as a production date and yeas of use), and the intervention effect information 102B becomes a movable range of a portion (for example, an arm) of the machine tool. In addition, the invention may be applied to stock investment. In this case, the intervention means information 101 becomes brands, the patient background information 102A becomes background information of investors (such as years of experience and investment amount), and the intervention effect information 102B becomes the number of shares held or sale price.

The invention is not limited to the above-described embodiments and includes various modifications and equivalent configurations within the spirit of the claims. For example, the above-described embodiments have been described in detail to make the invention easy to understand, and the invention is not necessarily limited to those having all the configurations described. A part of a configuration of a certain embodiment may be replaced with a configuration of another embodiment. A configuration of another embodiment may be added to a configuration of a certain embodiment. Further, another configuration may be added to, deleted from, or replaced with a part of a configuration of each embodiment.

Further, the configurations, functions, processing units, processing methods described above and the like may be implemented by a hardware by designing a part or all of them with an integrated circuit, for example, or may be implemented by a software by a processor interpreting and executing a program that implements each function.

Information such as a program, a table, and a file that implements each function can be stored in a storage device such as a memory, a hard disk, and a solid state drive (SSD), or a recording medium such as an integrated circuit (IC) card, an SD card, and a digital versatile disc (DVD).

Control lines and information lines indicate what is considered necessary for explanations, and do not necessarily indicate all control lines and information lines necessary for implementation. In practice, it may be considered that almost all the configurations are connected with each other.

What is claimed is:

1. An analyzer comprising:
a processor configured to execute a program; and
a storage device configured to store the program, wherein
the processor executes
a first calculation processing of calculating a first feature amount data group from an intermediate layer by inputting each training data of a training data group into a learning model which includes an input layer, one or more intermediate layers, and an output layer, and is learned based on the training data group assigned to the input layer and a correct answer data group assigned to the output layer,
a second calculation processing of calculating second feature amount data from the intermediate layer by inputting prediction target data of the learning model,
a search processing of searching specific first feature amount data similar to the second feature amount data calculated by the second calculation processing, from the first feature amount data group calculated by the first calculation processing, and
an extraction processing of extracting, from the training data group, specific training data, which is a calculation source of the specific first feature amount data searched by the search processing.

2. The analyzer according to the claim 1, wherein
in the search processing, the processor calculates a similarity between each first feature amount data of the first feature amount data group and the second feature amount data, and searches the specific first feature amount data from the first feature amount data group based on the similarity.

3. The analyzer according to the claim 2, wherein
in the search processing, the processor searches the first feature amount data whose similarity is equal to or greater than a predetermined threshold value as the specific first feature amount data.

4. The analyzer according to the claim 1, wherein
in the extraction processing, the processor extracts specific correct answer data corresponding to the specific training data from the correct answer data group.

5. The analyzer according to the claim 1, wherein
the processor executes a statistical processing of calculating a statistical value relating to the specific training data.

6. The analyzer according to the claim 1, wherein
the processor executes a statistical processing of calculating a statistical value relating to the specific correct answer data.

7. The analyzer according to the claim 1, wherein
the processor executes
a clustering processing of classifying the first feature amount data group into a plurality of clusters, and
a specifying processing of specifying an affiliation cluster of the second feature amount data from the plurality of clusters, and
in the search processing, the processor searches specific first feature amount data similar to the second feature amount data calculated by the second calculation processing, from the affiliation cluster specified by the specifying processing.

8. The analyzer according to the claim 1, wherein
the processor executes
a clustering processing of classifying the first feature amount data group into a plurality of clusters,
a generation processing of generating a prediction model based on training data which is a calculation source of first feature amount data in the cluster and correct answer data corresponding to the training data, for each of the plurality of clusters classified by the clustering processing,
a specifying processing of specifying an affiliation cluster of the second feature amount data from the plurality of clusters,
an acquisition processing of acquiring a prediction model of the affiliation cluster specified by the specifying processing from a plurality of prediction models generated by the generation processing, and
an output processing of outputting prediction result data by inputting the prediction target data in a prediction model acquired by the acquisition processing.

9. The analyzer according to the claim 1, wherein
each of the training data of the training data group and the prediction target data are first data strings indicating suitability of a plurality of different service attributes in a medical service, and each of the correct answer data of the correct answer data group is a second data string indicating information on a patient to which the medical service of the first data string is applied for the training data.

10. The analyzer according to the claim 9, wherein
the plurality of different service attributes includes a change from a first service attribute to a second service attribute.

11. The analyzer according to the claim 1, wherein
each of the training data of the training data group and the prediction target data are first data strings indicating suitability of a plurality of different types of medical services, and the correct answer data is a second data string indicating information on a patient to which the medical service of the first data string is applied for each of the correct answer data of the training data group.

12. An analysis method executed by an analyzer,
the analyzer including:
a processor configured to execute a program; and
a storage device configured to store the program,
the analysis method comprising:
executed by the processor
a first calculation processing of calculating a first feature amount data group from an intermediate layer by inputting each training data of a training data group into a learning model which includes an input layer, one or more intermediate layers, and an output layer, and is learned based on the training data group assigned to the input layer and a correct answer data group assigned to the output layer;
a second calculation processing of calculating second feature amount data from the intermediate layer by inputting prediction target data of the learning model;
a search processing of searching specific first feature amount data similar to the second feature amount data calculated by the second calculation processing, from the first feature amount data group calculated by the first calculation processing; and
an extraction processing of extracting, from the training data group, specific training data, which is a calculation source of the specific first feature amount data searched by the search processing.

13. An analysis program for causing a processor to execute
a first calculation processing of calculating a first feature amount data group from an intermediate layer by inputting each training data of a training data group into a learning model which includes an input layer, one or more intermediate layers, and an output layer, and is learned based on the training data group assigned to the input layer and a correct answer data group assigned to the output layer,
a second calculation processing of calculating second feature amount data from the intermediate layer by inputting prediction target data of the learning model,
a search processing of searching specific first feature amount data similar to the second feature amount data calculated by the second calculation processing, from the first feature amount data group calculated by the first calculation processing, and
an extraction processing of extracting, from the training data group, specific training data, which is a calculation source of the specific first feature amount data searched by the search processing.
